(19) United States
(12) Patent Application Publication
(10) Pub. No.: US 2018/0369154 A1
(43) Pub. Date: Dec. 27, 2018

(54) ACID-RESISTANT CAPSULE

(71) Applicant: GUANG DE LI CAPSULES CO., LTD., Taichung (TW)

(72) Inventor: Yueh Hua TSAI, Taichung (TW)

(21) Appl. No.: 15/816,440

(22) Filed: Nov. 17, 2017

(30) Foreign Application Priority Data
Jun. 22, 2017 (TW) 106209076

(51) Int. Cl. A61K 9/48

(52) U.S. Cl. CPC A61K 9/4808; A61K 9/4866; A61K 9/4891

(57) ABSTRACT

An acid-resistant capsule includes a shell body, a cap, and a coating layer; wherein the shell body and the cap are telescopically engaged together and the coating layer coated on the shell body is between the shell body and the cap. The shell body has a compartment and a first open end, the compartment is inside the shell body, and the first open end communicates with the compartment.

### CROSS-REFERENCE TO RELATED APPLICATION

Pursuant to 35 U.S.C. § 119(a), this application claims the benefit of the priority to Taiwan Patent Application No. 106209076 filed Jun. 22, 2017. The content of the prior application is incorporated herein by its entirety.

### BACKGROUND OF THE INVENTION

### 1. Field of the Invention

The present invention relates to a capsule, especially to an acid-resistant capsule.

### 2. Description of the Prior Arts

There are sorts of conventional capsules designed to enclose drugs or health products. The capsules are allowed to reach the stomach via oral administration and be dissolved by the gastric acid, so that the contents in the capsule are released into the digestive tract and then be absorbed by intestine. With reference to FIG. 8, U.S. Pat. No. 4,138,013 discloses a conventional capsule structure comprising a shell body 70 and a cover body 80. The shell body 70 has a compartment 71 inside to be filled with a substance 8 such as active composition powder. The cover body 80 is fitted or covered to the shell body 70 to keep the substance 8 in the compartment 71 of the shell body 70.

However, the seam between the cover body 80 and the shell body 70 is not tight and not resistant to extrusion. When the capsule is into the stomach, the gastric acid will quickly penetrate into the compartment of the capsule from the seam, and erode the capsule and the substance 8. As the substance 8 is destroyed by gastric acid before entering the intestinal tract, the intestinal absorption rate of the substance 8 is greatly reduced.

With reference to FIG. 9, U.S. Pat. No. 4,478,658 discloses another conventional capsule structure, using a banding 81 to seal the seam between the shell body 70 and the cover body 80. However, the seam is not completely sealed, the gastric acid will quickly penetrate into the compartment 71 of the shell body 70 of the capsule from the seam, and thus it will also erode the substance 8 of the capsule and the capsule itself.

With reference to FIG. 10, TW 414232 discloses another conventional capsule structure, wherein the seam of the capsule is entirely sealed with the banding 81 to prevent the gastric acid penetrating from the seam into the compartment 71 of the shell body 70 of the capsule. However, the sealing step must be performed after the shell body and the cover body are engaged together to coat or dispose the banding on the seam between the shell body and the cover body, and thus the sealing step is complicated and time consuming.

### SUMMARY OF THE INVENTION

To overcome the shortcomings of the above-mentioned, the objective of the present invention is to provide an acid-resistant capsule, which is easily manufactured with its assembly simplified.

The present invention provides an acid-resistant capsule, comprising a shell body, a cap, and a coating layer. The shell body and the cap are telescopically engaged together and the coating layer is interposed between the shell body and the cap. The shell body has a compartment and a first open end, the compartment is inside the shell body to be filled with a substance, and the first open end communicates with the compartment. The coating layer, which is between the shell body and the cap, makes the shell body and the cap tightly telescopically engaged together, so that the substance can be maintained in the compartment of the shell body.

Preferably, the coating layer is coated on the outer side of the shell body near the first open end of the shell body, such that the coating layer is interposed between the shell body and the cap.

Preferably, the coating layer is coated on the outer surface and the inner surface of the shell body, such that the coating layer is not only interposed between the shell body and the cap but also covers the shell body.

Preferably, the coating layer is coated on the inner surface of the cap, such that the coating layer is not only interposed between the shell body and the cap but also covers the cap.

Preferably, the coating layer is coated on the outer surface and the inner surface of the cap, such that the coating layer is not only interposed between the shell body and the cap but also covers the cap.

Preferably, the thickness of the coating layer is 0.01 mm to 0.15 mm.

Preferably, the acid-resistant capsule further comprises an outer clothing which is coated on the outer surfaces of the shell body and the cap that are engaged together.

Preferably, the coating layer is selected from the group consisting of ethyl cellulose, methacrylic acid—ethyl acrylate copolymer and hydroxypropyl methylcellulose.

More preferably, the content of ethyl cellulose in the coating layer is 5% to 35%.

More preferably, the coating layer comprises an inner layer and an outer layer, the inner layer is between the outer layer and the surface of the shell body or the cap, wherein the material of the inner layer of the coating layer is methacrylic acid—ethyl acrylate copolymer, and the material of the outer layer of the coating layer are ethyl cellulose and hydroxypropyl methylcellulose.

More preferably, the outer layer of the coating layer includes 35% to 70% of ethyl cellulose and 30% to 65% of hydroxypropyl methylcellulose.

The advantages of the present invention are as follows: the compartment of the shell body can be filled with the active composition, such as drug or health product, and then is assembled with the cap to complete the acid-resistant capsule, that is, the acid-resistant capsule only needs to be processed with the filling step. The coating layer makes the shell body and the cap securely engaged with each other, and thus the acid-resistant capsule of the present invention has the effect of resisting gastric acid erosion without the sealing step.

### BRIEF DESCRIPTION OF THE DRAWINGS

FIG. 1 is a perspective view of the first embodiment of the acid-resistant capsule of the present invention.

FIG. 2 is an exploded view of the first embodiment of the acid-resistant capsule of the present invention.

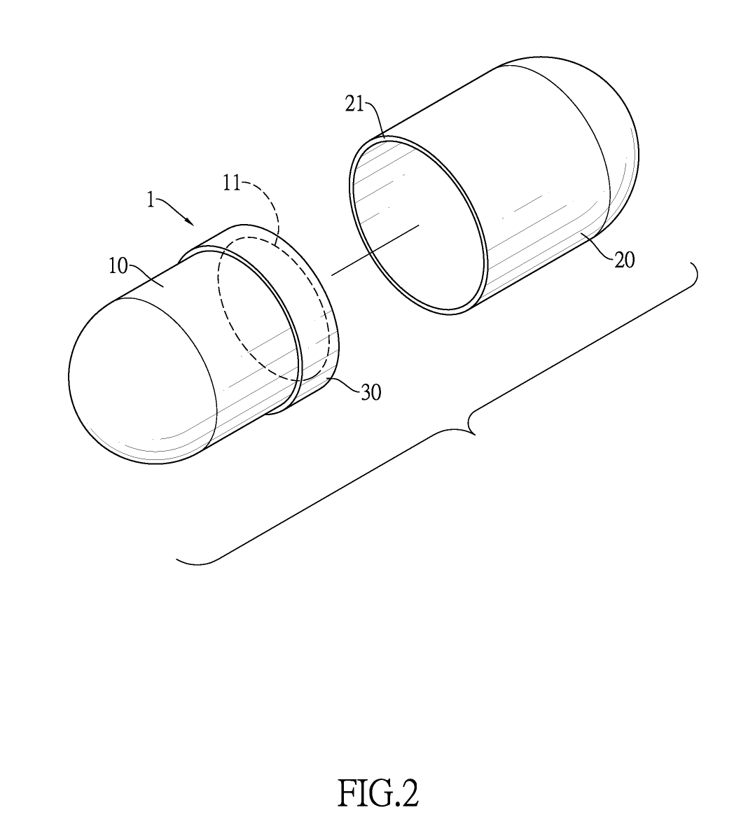

FIG. 3 is a cross-sectional schematic view across line 6-6 in FIG. 1.

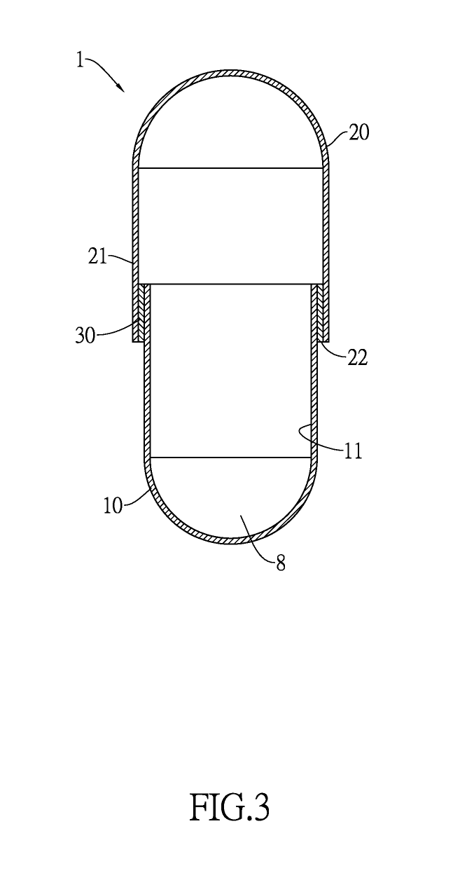

FIG. 4 is a cross-sectional schematic view across line 3-3 in FIG. 2.

FIG. 5 is a cross-sectional schematic view of the second embodiment of the acid-resistant capsule of the present invention.

FIG. 6 is a cross-sectional schematic view of the third embodiment of the acid-resistant capsule of the present invention.

FIG. 7 is a cross-sectional schematic view of the fourth embodiment of the acid-resistant capsule of the present invention.

FIG. 8 is a perspective view of a capsule of the prior art.

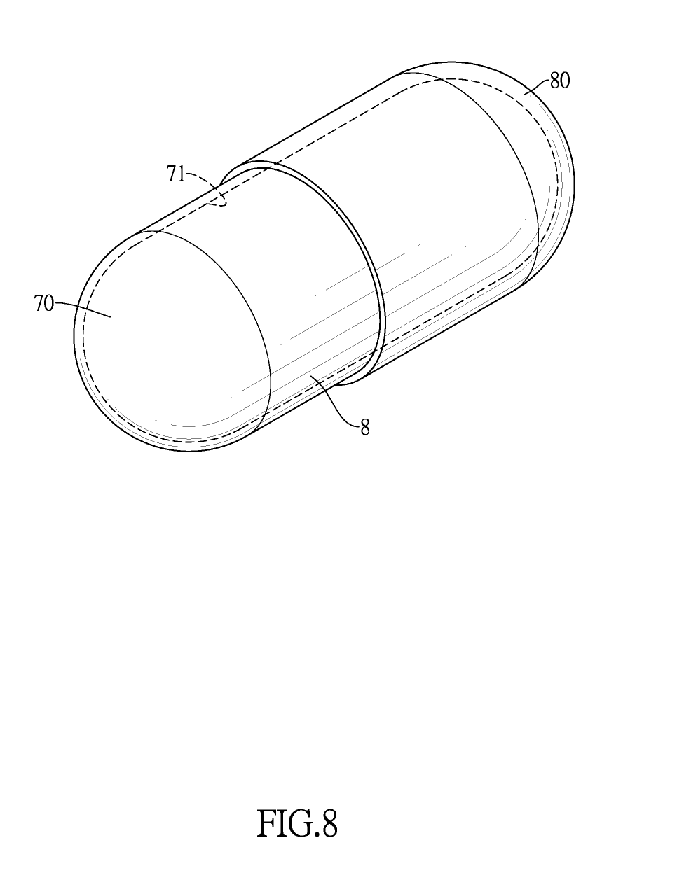

FIG. 9 is another perspective view of a capsule of the prior art.

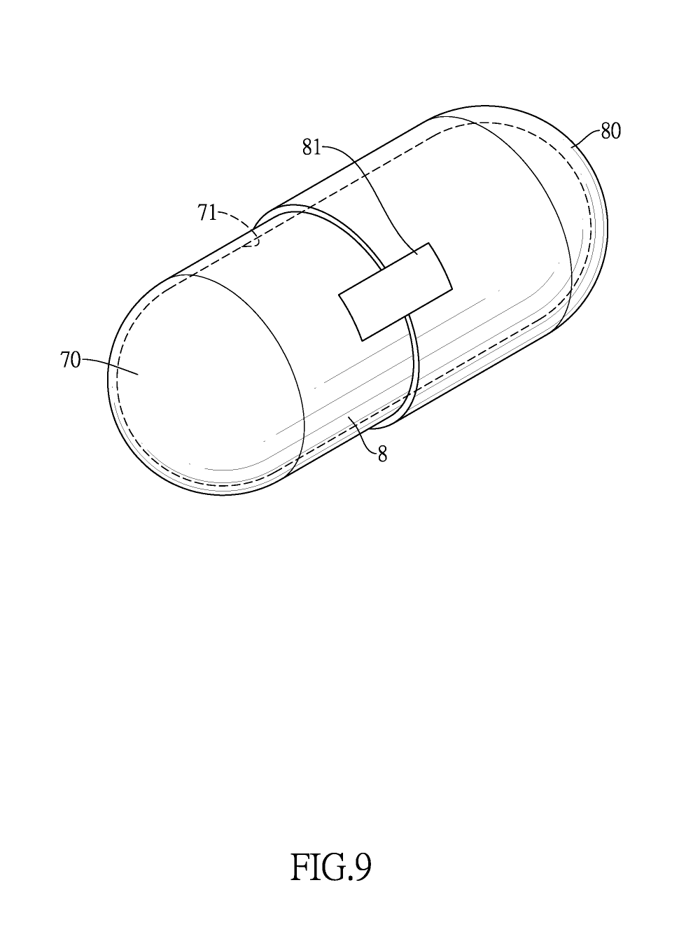

FIG. 10 is still another perspective view of a capsule of the prior art.

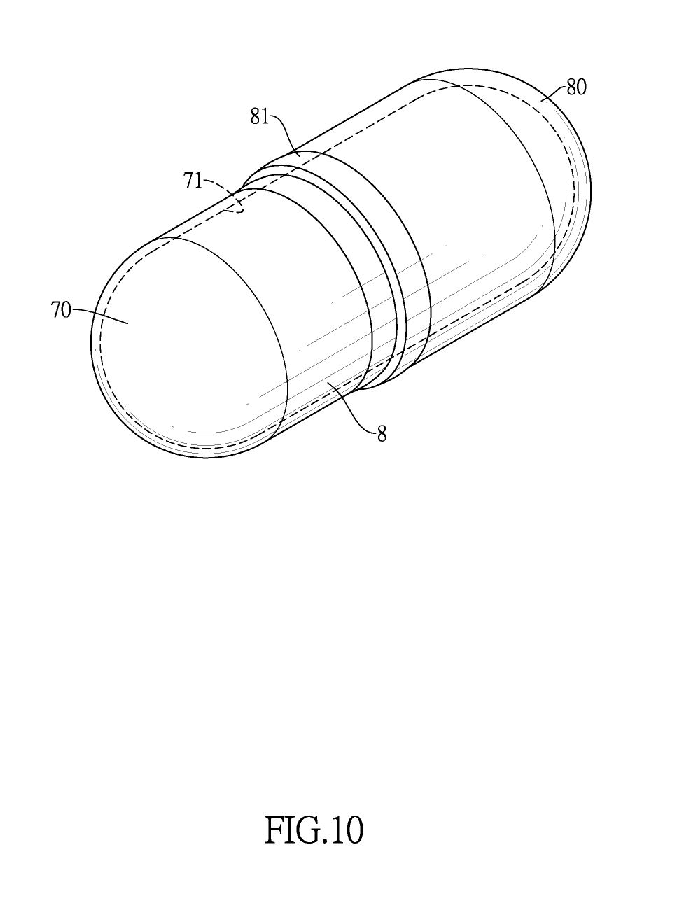

### DETAILED DESCRIPTION OF THE PREFERRED EMBODIMENTS

Other objectives, advantages and novel features of the invention will become more apparent from the following detailed description when taken in conjunction with the accompanying drawings.

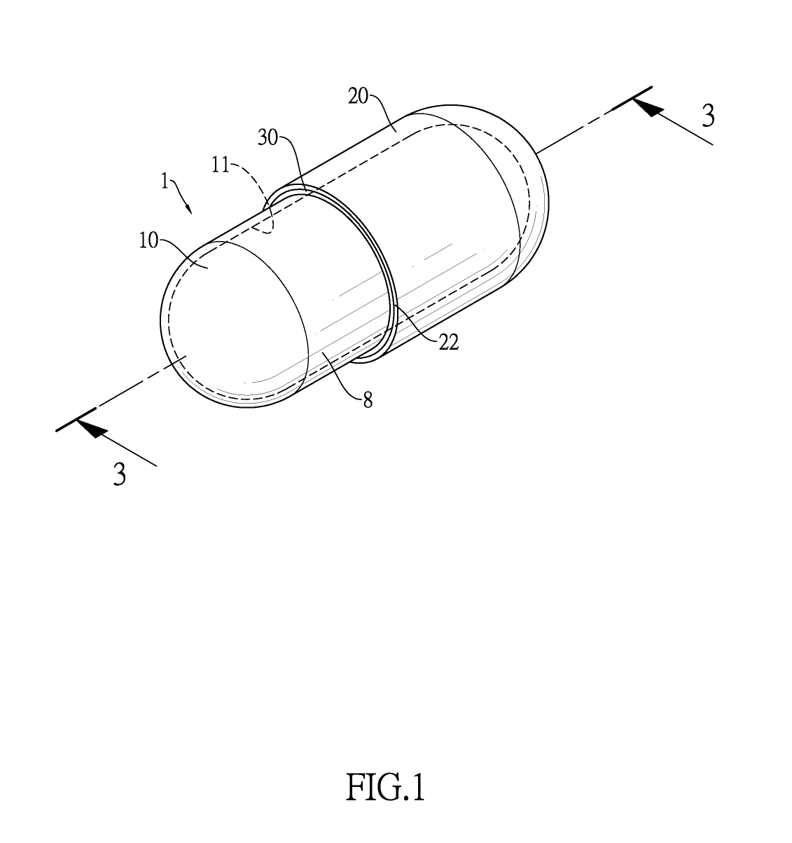

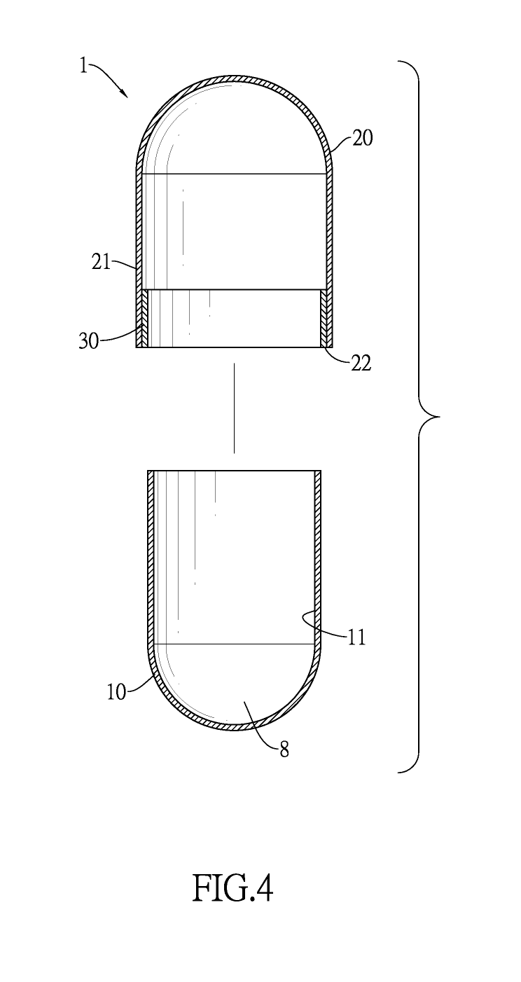

With reference to FIGS. 1 and 4, the first embodiment of the acid-resistant capsule 1 in accordance with the present invention comprises a shell body 10, a cap 20, and a coating layer 30. The shell body 10 and the cap 20 are telescopically engaged together, and the coating layer 30 coated on the shell body 10 is interposed between the shell body 10 and the cap 20. The shell body 10 has a compartment 11 and a first open end 12, the compartment 11 is inside the shell body 10 to be filled with substances, such as active composition powder, and the first open end 12 communicates with the compartment 11. The cap 20 has a second open end 21, which can engage with the first open end 12 of the shell body 10, and then a seam 22 is formed by the shell body 10 and the cap 20 that are engaged. The coating layer 30 between the shell body 10 and the cap 20 makes the shell body 10 and the cap 20 tightly telescopically engaged together, so that the substance can be maintained in the compartment 11 of the shell body 10.

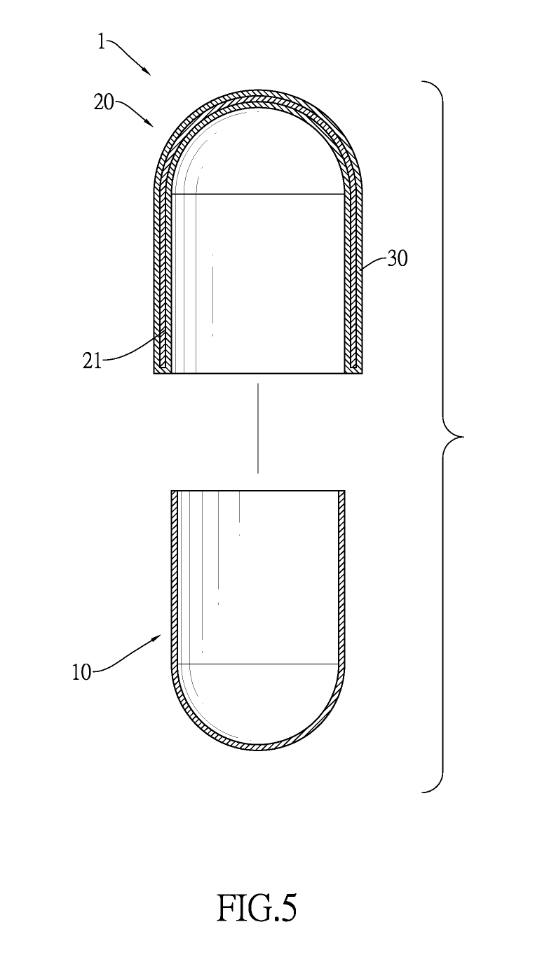

With reference to FIG. 5, the second embodiment of the acid-resistant capsule 1 is similar to the first embodiment, and the difference is that the coating layer 30 is coated on the outer surface and the inner surface of the cap 20.

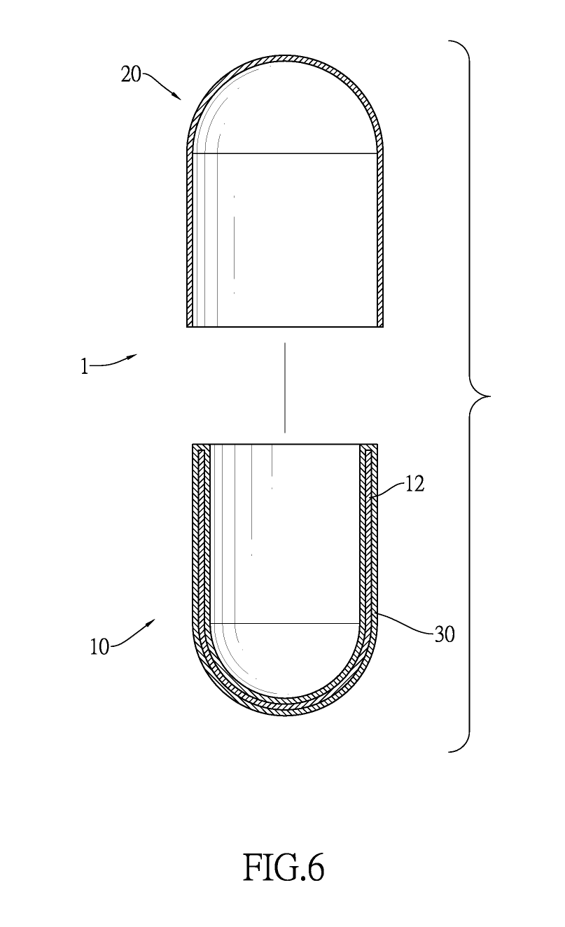

With reference to FIG. 6, the third embodiment of the acid-resistant capsule 1 is similar to the first embodiment, and the difference is that the coating layer 30 is coated on the outer surface and the inner surface of the shell body 10.

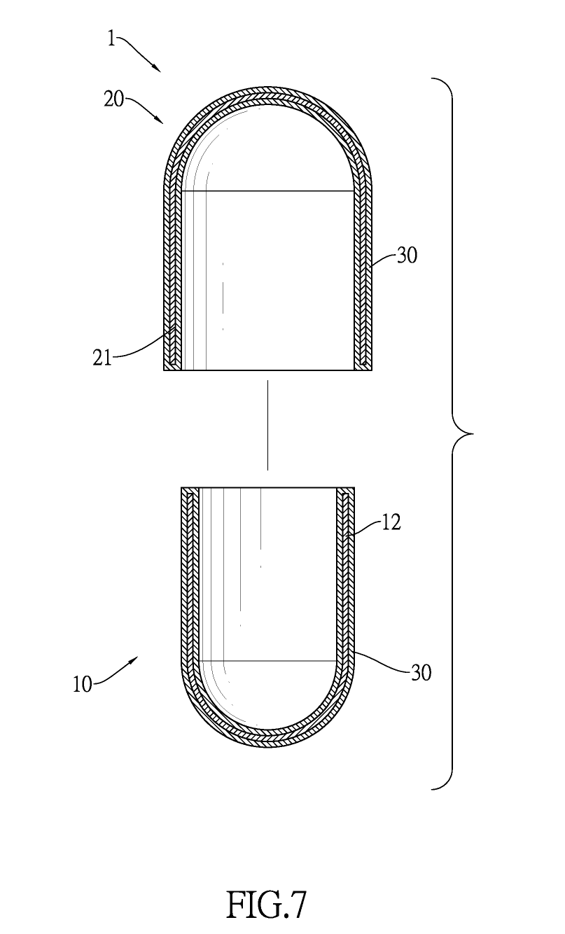

With reference to FIG. 7, the fourth embodiment of the acid-resistant capsule 1 is similar to the second embodiment, and the difference is that the coating layer 30 is further coated on the outer surfaces and the inner surfaces of the shell body 10 and the cap 20.

In another embodiment, the material of the coating layer 30 comprises ethyl cellulose (EC), methacrylic acid-ethyl acrylate copolymer, and hydroxypropyl methylcellulose (HPMC), and the content of EC in the coating layer 30 is 5% to 35%.

In another embodiment, the coating layer comprises an inner layer and an outer layer, the inner layer is between the outer layer and the surface of the shell body or the cap, wherein the material of the inner layer of the coating layer is methacrylic acid—ethyl acrylate copolymer, and the material of the outer layer of the coating layer are ethyl cellulose and hydroxypropyl methylcellulose. Further, the outer layer of the coating layer includes 35% to 70% of ethyl cellulose and 30% to 65% of hydroxypropyl methylcellulose.

The ethyl cellulose and methacrylic acid-ethyl acrylate copolymer can be adjusted according to different desires, such as the capsule would like to be absorbed at duodenum. In another embodiment, the acid-resistant capsule 1 could be further processed with an enteric coating step after the filling and assembling steps.

Even though numerous characteristics and advantages of the present invention have been set forth in the foregoing description, together with details of the structure and features of the invention, the disclosure is illustrative only. Changes may be made in the details, especially in matters of contour, size, and arrangement of parts within the principles of the invention to the full extent indicated by the broad general meaning of the terms in which the appended claims are expressed.

What is claimed is:

1. An acid-resistant capsule, comprising:
a shell body having
a compartment, which is inside the shell body, to be filled within a substance; and
a first open end, which communicates with the compartment; and
a cap, which is telescopically engaged with the shell body; and
a coating layer interposed between the shell body and the cap.

2. The acid-resistant capsule according to claim 1, wherein the coating layer is coated on the outer side of the shell body near the first open end of the shell body.

3. The acid-resistant capsule according to claim 1, wherein the coating layer is coated on the outer surface and the inner surface of the shell body.

4. The acid-resistant capsule according to claim 1, wherein the coating layer is coated on the inner surface of the cap.

5. The acid-resistant capsule according to claim 1, wherein the coating layer is coated on the outer surface and the inner surface of the cap.

6. The acid-resistant capsule according to claim 1, wherein the thickness of the coating layer is 0.01 mm to 0.15 mm.

7. The acid-resistant capsule according to claim 1, wherein the acid-resistant capsule further comprises an outer clothing which is coated on the outer surfaces of the shell body and the cap that are engaged together.

8. The acid-resistant capsule according to claim 1, wherein a material of the coating layer is ethyl cellulose, methacrylic acid—ethyl acrylate copolymer and hydroxypropyl methylcellulose.

9. The acid-resistant capsule according to claim 8, wherein the content of ethyl cellulose in the coating layer is 5% to 35%.

10. The acid-resistant capsule according to claim 6, wherein the coating layer comprises an inner layer and an outer layer, the inner layer is between the outer layer and the surface of the shell body or the cap, wherein the material of the inner layer of the coating layer is methacrylic acid—ethyl acrylate copolymer, and the material of the outer layer of the coating layer are ethyl cellulose and hydroxypropyl methylcellulose.

11. The acid-resistant capsule according to claim 10, wherein the outer layer of the coating layer includes 35% to 70% of ethyl cellulose and 30% to 65% of hydroxypropyl methylcellulose.
